(19) United States
(12) Patent Application Publication
(10) Pub. No.: US 2008/0190174 A1
(43) Pub. Date: Aug. 14, 2008

(54) Sensor Module Package

(76) Inventors: Berend Jan Kooi, Enschede (NL); Gerrit Hendrik Nijmeijer, Enschede (NL); Edward Gruterink, Aadorp (NL); Tim Tiek, Enschede (NL); Huub Gerard Toneman, Enschede (NL)

Correspondence Address: SENSATA TECHNOLOGIES, INC., 529 PLEASANT STREET, MS B-1, ATTLEBORO, MA, 02703, US

(21) Appl. No.: 11/913,782

(22) PCT Filed: May 10, 2005
(86) PCT No.: PCT/NL2005/000350

§ 371 Date: Nov. 7, 2007

(51) Int. Cl. G01N 33/00

(52) U.S. Cl. 73/31.01

(57) ABSTRACT

Sensor module package having a housing element (3) with an opening (4) to a sensor cavity (6) for allowing outside air to reach a sensor device (1) positioned in the sensor cavity (6) for sensing a characteristic of the outside air. Sensor electronics (9) are connected to the sensor device (1) for providing an output signal. The sensor device (1) and sensor electronics (9) are positioned on a substrate (2). The sensor cavity (6) is formed by at least a cavity part of the substrate (2), at least a part of the housing element (3) and the opening (4). A cover member (11) is provided with a mechanical firming element (13) for mounting the sensor module package (10) in a vehicle.

### FIELD OF THE INVENTION

The present invention relates to a sensor module package comprising a housing element with an opening to a sensor cavity for allowing outside air to reach a sensor device positioned in the sensor cavity for sensing a characteristic of the outside air, sensor electronics connected to the sensor device for providing an output signal, the sensor device and sensor electronics being positioned on a substrate.

### PRIOR ART

A gas detecting device for a vehicle has been disclosed in European patent application EP-A-0 712 745. The device comprises a printed circuit board on which a gas sensor element and processing electronics are positioned, and a housing for accommodating the printed circuit board. The housing comprises a sensor cavity in which the sensing surface of the gas sensor element protrudes, and an air labyrinth for transporting outside air from an inlet opening, via a filter to the sensor cavity, and onwards to an outlet opening. The sensor cavity is entirely defined by the housing, the filter outlet surface and the sensor element surface. As a result, the assembly of the gas detecting device is quite cumbersome and complex.

A pollutant probe is disclosed in EP-A-0 779 170. This device comprises a printed circuit board with evaluation electronics mounted in a sensor housing. The sensor device is positioned on a tongue connected to the printed circuit board, extending outward from the sensor housing. A sensor cavity is formed by positioning a housing cover over the entire tongue with sensor. The housing cover needs to be positioned in order to protect the sensor device on the tongue. In all, assembly of this pollutant probe is also complex.

### SUMMARY OF THE INVENTION

The present invention seeks to provide an air quality sensor module, which is easy to assemble, and in which the sensor is also protected from moisture or particles in the outside air.

According to the present invention, a sensor module package according to the preamble defined above is provided, in which the sensor cavity is formed by at least a cavity part of the substrate, at least a part of the housing element and the opening. By using part of the substrate (the cavity part) as a wall of the sensor cavity, e.g. the bottom wall, and the sensor housing element and the opening as further borders of the sensor cavity, the assembly of the sensor module package is very simple and the sensor package module can be manufactured very efficiently.

In a further embodiment, the sensor device is positioned on the cavity part of the substrate, to allow direct interaction of the outside air with the sensitive surface of the sensor device. This may e.g. be accomplished using surface mount technique to attach the sensor device on the substrate (such as a printed circuit board). No further mounting elements or positioning elements are then needed for the sensor device. When a sensor device is used which requires an elevated operating temperature, the sensor device itself is advantageously provided with a thermal isolating barrier, in order to be able to attach the sensor device directly on the substrate. This may be accomplished using a MEMS sensor device, in which the sensitive surface is provided on a free membrane.

A sealing member is provided between the housing element and the substrate in a further embodiment, in order to allow an effective sealing of the sensor cavity, such that the outside air can only reach this sensor cavity. To allow only outside air to enter the sensor cavity, the opening is provided with a semi-permeable membrane in a further embodiment, which membrane allows outside air to enter the sensor cavity. Water vapor and particles can be effectively stopped from entering the sensor cavity, thus protecting the sensor device from possible damage. The semi-permeable membrane is e.g. made of a Gore-Tex kind of material.

In a further embodiment, the sensor electronics are positioned on a side of the substrate opposing the sensor cavity. The sensor electronics are thus protected from outside influences due to the outside air in the sensor cavity. Connections between the sensor device and the processing electronics may be provided in conventional manner, e.g. using vias.

In an advantageous embodiment, the housing element comprises locking members for locking the substrate in the housing element. By locking the substrate in the housing element, a semi-product is obtained. This allows e.g. to assemble the substrate (with sensor device) in the housing element in one place, and perform calibration of the semi-product in another place.

The sensor module package further comprises a cover member in a further embodiment, in which the cover member is arranged to seal off an electronics cavity formed by the housing element, the substrate and the cover member. The cover member may be of the same material as the housing element, and the cover member may be attached to the housing element using e.g. laser welding, gluing, etc.

To assure a proper sealed of sensor cavity in an assembled state of the sensor module package, the cover member comprises extending members for pressing the substrate in the housing element. The extending members may be pins or otherwise formed extensions of the cover member, and advantageously form an integral part of the cover member (e.g. by injection molding of the cover member with extending members).

To be able to mount the sensor module package in a vehicle, the cover member advantageously comprises a mechanical fixing element for mounting the sensor module package in a vehicle. Such a mechanical fixing element may be of a standardized form, to allow mounting the sensor package module in a wide variety of vehicle types.

### SHORT DESCRIPTION OF DRAWINGS

The present invention will be discussed in more detail below, using a number of exemplary embodiments, with reference to the attached drawings, in which

FIG. 1 shows a cross sectional view of an embodiment of the sensor module package according to the present invention.

### DETAILED DESCRIPTION OF EXEMPLARY EMBODIMENTS

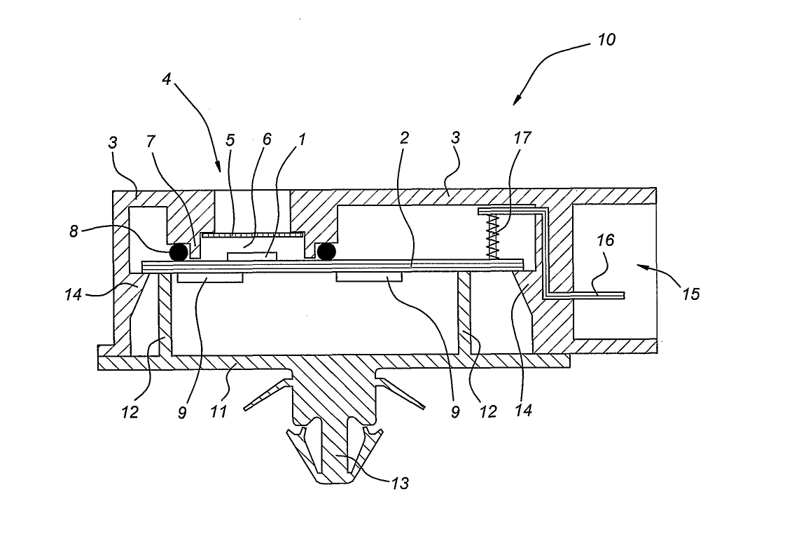

An embodiment of the present sensor module package 10 is shown in a schematic cross sectional view in FIG. 1. The sensor module package 10 comprises a sensor device 1, which is sensitive to predetermined substances in outside air, e.g. carbon monoxide, nitrogen oxides, hydrocarbons or volatile organic compounds. The sensor device 1 may comprise a single sensor element, but may also be provided with multiple sensor elements, e.g. one for each substance to be detected.

The sensor module package 10 further comprises a housing element 3, e.g. made from a plastic mould. The housing element 3 comprises an opening 4, defined by side walls 7. In this embodiment the opening 4 is circular, but other shapes may be used, such as rectangular or square. The opening is covered by a membrane 5, which is intended to protect the inside of the sensor module 10 from moisture and particles contained in the outside air. The membrane may e.g. be made of a semi-permeable material, which allows gasses, such as outside air, to pass, but prevents water and dust particles from entering the sensor module 10.

The sensor module package 10 comprises a substrate 2, such as a printed circuit board. On the substrate 2 the sensor device 1 is positioned on a first side, e.g. using known gluing or soldering techniques. On the other side of the substrate 2, processing electronics 9 are positioned, which are connected to the sensor device 1 using known techniques, e.g. using vias in the substrate 2. When a sensor device 1 is used which requires an elevated operating temperature, the sensor device 1 itself is advantageously provided with a thermal isolating barrier, in order to be able to attach the sensor device 1 directly on the substrate 2.

The substrate 2 forms a sensor cavity 6 in co-operation with the side walls 7 of the housing element 3 and the membrane 5. The side walls 7 are adapted to provide an air tight connection with the substrate 2. In the shown embodiment, the air tight sealing between side walls 7 and substrate 2 is accomplished using a sealing element 8 in the form of an O-ring, e.g. made of rubber or silicone material. As the substrate 2 in co-operation with the housing element 3 provides for the sensor cavity 6 in the sensor module package 10, assembly of the sensor module package 10 is very simple, and the sensor module package 10 may be manufactured very efficiently.

During the manufacturing process, the sensor module package 10 may be assembled in stages. First, the housing element 3 is manufactured, e.g. using injection molding or other casting techniques. In the housing element 3, a connector part 15 is integrated. The connector part 15 is further provided with terminals 16 (one shown in FIG. 1, but more may be present, e.g. three terminals 16), which are e.g. positioned using predefined mounting holes in the housing element 3. An electrical connection between the terminal 16 and substrate is provided by a conductive spring 17 which in assembled state is pressed against a connecting pad on the substrate 2 for providing the electrical connection in this embodiment. In alternative embodiments, the connection may e.g. be obtained using a pin and hole connection. The connector part 15 is preferably formed as a standard type of connector, e.g. a standard automotive connector such as an MQS connector (Micro Quadlock System).

The opening 4 in the housing element 3 is then covered by the membrane 5, e.g. using a welding technique or a glue, and the O-ring 8 is positioned around the side walls 7 as indicated in FIG. 1. Subsequently, the pre-manufactured substrate 2, complete with sensor device 1 and processing electronics 9 is positioned in the housing element 3, such that the sensor cavity 6 is formed automatically. The substrate 2 may, in an advantageous embodiment, be fixed in the housing element 3 using the fixing means 14, which are an integral part of the housing element 3. The fixation of the substrate 2 may be accomplished by simply pressing the substrate 2 beyond the extending parts of the fixing means 14 (snap fit). Alternatively, the fixing means 14 are formed in order to clamp the substrate 2 in the housing element 3.

The sensor module package 10 is now ready for calibration, which is easily achievable, as the processing electronics are readily reachable from the open side of the housing element 3. After calibration the sensor module package 10 is provided with a cover element 11, such that also the processing electronics 9 are protected against damage and external influences. The cover element 11 is provided with pins 12, which provide an additional fixation of the substrate 2 in the housing element 3. The cover element 11 may further be provided with mounting means 13, which allow for fixing the sensor module package 10 in e.g. a vehicle. Alternatively, the housing element 3 is provided with mounting means 13 on the front side (gas opening side) of the sensor module package 10, e.g. in the form of a bayonet connection. As a final step, the cover element 11 and housing element 3 are attached to each other, e.g. by laser welding or gluing, to provide a completely closed sensor module package 10 ready for use.

1. A sensor module package comprising a housing element with an opening to a sensor cavity for allowing outside air to reach a sensor device positioned in the sensor cavity for sensing a characteristic of the outside air, sensor electronics connected to the sensor device for providing an output signal, the sensor device and sensor electronics being positioned on a substrate, characterized in that the sensor cavity is formed by at least a cavity part of the substrate, at least a part of the housing element and the opening and the sensor electronics are positioned outside of the cavity.

2. A sensor module package according to claim 1, in which the sensor device is positioned on the cavity part of the substrate.

3. A sensor module package according to claim 1, in which a sealing member is provided between the housing element and the substrate.

4. A sensor module package according to claim 1, in which opening is provided with a semi-permeable membrane allowing outside air to enter the sensor cavity.

5. A sensor module package according to claim 1, in which the sensor electronics are positioned on a side of the substrate opposing the sensor cavity.

6. A sensor module package according to claim 1, in which the housing element comprises locking members for locking the substrate in the housing.

7. A sensor module package according to claim 1, in which the sensor module package further comprises a cover member, arranged to seal off an electronics cavity formed by the housing element, the substrate and the cover member.

8. A sensor module package according to claim 7, in which the cover member comprises extending members for pressing the substrate in the housing element.

9. A sensor module package according to claim 7, in which the cover member comprises a mechanical fixing element for mounting the sensor module package in a vehicle.
